(19) United States
(12) Patent Application Publication
(10) Pub. No.: US 2020/0264454 A1
(43) Pub. Date: Aug. 20, 2020

(54) EYEGLASSES WITH BIO-SIGNAL SENSORS

(71) Applicant: SAFILO SOCIETÀ AZIONARIA FABBRICA ITALIANA LAVORAZIONE OCCHIALI S.P.A., Padova (IT)

(72) Inventors: Samuel Thomas MACKENZIE, Toronto (CA); Graeme Daniel MOFFAT, Toronto (CA); Christopher Allen AIMONE, Toronto (CA); Locillo (Lou) Giuseppe PINO, Cambridge (CA); Marta Joanna ZACHAROWSKA, Toronto (CA); Derek Geoffrey PYNE, Toronto (CA)

(73) Assignee: SAFILO SOCIETÀ AZIONARIA FABBRICA ITALIANA LAVORAZIONE OCCHIALI S.P.A., Padova (IT)

(21) Appl. No.: 16/067,881

(22) PCT Filed: Dec. 29, 2016
(86) PCT No.: PCT/EP2016/082868

§ 371 Date: Jul. 3, 2018

Related U.S. Application Data
(60) Provisional application No. 62/275,061, filed on Jan. 5, 2016.
(60) Provisional application No. 62/326,563, filed on Apr. 22, 2016.

(51) Int. Cl. G02C 11/00; G02C 5/12; G02C 5/14; G02C 5/22; A61B 5/0478; A61B 5/00

(52) U.S. Cl. CPC G02C 11/10; G02C 5/12; G02C 5/143; A61B 2562/0215; A61B 5/0478; A61B 5/6803; G02C 5/22

(57) ABSTRACT

An eyeglass frame includes a front portion (130) for holding two lenses, an assembly for attaching to a signal pod (110), side arms (140, 150) connected to the front portion (130), a nose assembly (160) connected to the front portion (130) and also includes nose contacts (162, 164) for supporting the front portion (130) on the user's nose. The nose contact (162, 164) is electrically connected to the signal pod (110), and an adjustable ear piece (1402) connected to the side arms and includes head contacts in contact with a user's head and ears. The head contacts are electrically connected to the side arms (140, 150) and the signal pod (110), the adjustable ear piece being formable or malleable to conform to the user's head and ears.

### TECHNICAL FIELD

Embodiments described herein relate to wearable devices. Embodiments described herein relate more particularly to an eyeglass with bio-signal (i.e. EEG) sensors.

### TECHNOLOGICAL BACKGROUND

There is a need for devices for incorporating bio-signal sensors into eyewear.

### DESCRIPTION OF THE INVENTION

The scope of the present application is not intended to be limited to the particular embodiments and/or aspects of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Further, as can be understood, the examples described and illustrated herein are intended to be exemplary only.

In a first aspect, there is provided an eyeglass frame for connecting to a signal pod comprising: a front portion for holding two lenses; an assembly for attaching to the signal pod; a first side arm connected to the front portion and having a first side arm contact in contact with a user's head where the first side arm contact is electrically connected to a first side arm point; a second side arm connected to the front portion opposite the first side arm and having a second side arm contact in contact with the user's head where the second side arm contact is electrically connected to a second side arm point; a nose assembly connected to the front portion and having a first nose contact and a second nose contact for supporting the front portion on the user's nose where the first nose contact is electrically connected to a first nose point and the second nose point. The first side arm contact, the second side arm contact, the first nose contact and the second nose contact are electrically isolated from each other over the eyeglass frame. This is an example embodiment and there may be more contacts in other example embodiments.

In another aspect, the first side arm and the second side arm of the eyeglass frame are pivotally hinged to the front portion.

In another aspect, the first side arm point, the second side arm point, the first nose point, and the second nose point of the eyeglass frame are all on one of the first side arm, the second side arm, and the front portion for connecting to the signal pod.

In another aspect, the eyeglass frame further comprises one of pod slot and ferro metal slot for attachment with the signal pod.

In another aspect, there is provided a signal pod for connecting to an eyeglass frame comprising: an assembly for attaching to the eyeglass frame; a first pod contact for electrically connecting with a first side arm point of the eyeglass frame; a second pod contact for electrically connecting with a second side arm point of the eyeglass frame; a third pod contact for electrically connecting with a first nose point of the eyeglass frame; a fourth pod contact for electrically connecting with a second nose point of the eyeglass frame; and an electrical module to measure electrical potentials between the first pod contact, the second pod contact, the third pod contact, and the fourth pod contact and to transmit such measurements to a computing device for processing and further output. This is an example embodiment. In other example embodiments there may be more than four pod contacts.

In another aspect, the first pod contact, the second pod contact, the third pod contact, and the fourth pod contact of the signal pod are spring loaded for contacting with the eyeglass frame.

In another aspect, the assembly for attaching the signal pod to the eyeglass frame comprises a clip for clipping onto a slot on the eyeglass frame.

In another aspect, the assembly for attaching the signal pod to the eyeglass frame comprises a magnet for magnetically attaching to a ferro metal slot on the eyeglass frame.

In another aspect there is provided an eyeglass frame integrating a signal pod comprising: an assembly for attaching to the signal pod; a side arm connected to the front portion and having a first side arm contact in contact with a first portion of a user's ear or head where the first side arm contact is electrically connected to a first side arm point, and having a second side arm contact in contact with a second portion of a user's ear or head where the second side arm contact is electrically connected to a second side arm point; wherein the first side arm contact and the second side arm contact are electrically isolated from each other over the eyeglass frame, and wherein the first side arm contact and the second side arm contact incorporate conductive ink printed on the side arm of the eyeglass frame, conductive rubber adhered or otherwise coupled to the side arm of eyeglass frame, and/or conductive fabric, metal, or another conductive material in order to facilitate detecting bio-signals produced by user and contour of the first portion of a user's ear and the second portion of a user's ear to ensure sufficient contact. The detection of the bio-signals may be carried out on the contour of the first portion of the user's ear and/or on the second portion of the user's ear.

In another aspect there is provided an eyeglass frame integrating a signal pod comprising: an assembly for attaching to the signal pod; a side arm connected to the front portion and having a first side arm contact in contact with a first portion of a user's ear or head where the first side arm contact is electrically connected to a first side arm point, and having a second side arm contact in contact with a second portion of a user's head where the second side arm contact is electrically connected to a second side arm point; wherein the first side arm contact and the second side arm contact are electrically isolated from each other over the eyeglass frame and connected to the assembly; and wherein the first side arm contact and the second side arm contact incorporate conductive ink printed on the side arm of the eyeglasses frame, conductive rubber adhered or otherwise coupled to the side arm of eyeglass frame, and/or conductive fabric, metal, or another conductive material in order to facilitate detecting bio-signals produced by user and contour of the first portion of a user's ear and the second portion of a user's ear to ensure sufficient contact. The detection of the bio-signals may be carried out on the contour of the first portion of the user's ear and/or on the second portion of the user's ear.

In another aspect there is provided an eyeglass frame integrating a signal pod comprising: an assembly for attaching to the signal pod; a nose assembly connected to the assembly and having a first nose contact and a second nose contact for supporting the front portion on a user's nose where the first nose contact is electrically connected to a first nose point and the second nose point; wherein the first nose contact and the second nose contact are electrically isolated from each other over the eyeglass frame and connected to the assembly; and wherein the first nose contact and the second nose contact incorporate conductive ink printed on the nose assembly of the eyeglass frame, conductive rubber adhered or otherwise coupled to the nose assembly of eyeglass frame, and/or conductive fabric, metal, or another conductive material in order to facilitate detecting bio-signals produced by user and contour of the user's nose to ensure sufficient contact. The detection of the bio-signals may be carried out on the contour of the user's nose.

In another aspect the eyeglass frame integrating a signal pod comprising temple electrodes and temple electrode springs attached to the eyeglass frame at a first inner surface of a first side arm of the eyeglass frame, and/or a second inner surface of a second side arm of the eyeglass frame

### DESCRIPTION OF THE FIGURES

FIG. 1A is a top view of an example of an eyeglass frame with an attached signal pod;

FIG. 1B is a rear view of the eyeglass frame with attached signal pod;

FIG. 2 is a top view of the eyeglass frame with two attached signal pods;

FIG. 3 is a side view of the eyeglass frame side arm and attachment points for the signal pod;

FIG. 4A is a side view of an example signal pod and the signal pod's attachment and communication means;

FIG. 4B is a top view of an example signal pod and the signal pod's attachment and communication means;

FIG. 5 is a block diagram depicting a generic computer device and a user interacting with the generic computer device;

FIG. 6A is a top view of an example signal pod and the signal pod's attachment and communication means;

FIG. 6B is a side view of an example signal pod and the signal pod's attachment and communication means;

FIG. 7 is a side view of an example signal pod and the signal pod's attachment and communication means attached to an example eyeglass frame arm;

FIG. 8A is a side view of an example eyeglass frame arm and attached bio-signal sensors;

FIG. 8B is a side view of an example eyeglass frame arm and attached bio-signals placed upon the ear of a user;

FIG. 9A is a rear view of an example eyeglass frame and attached bio-signal;

FIG. 9B is a top view of an example eyeglass frame and attached bio-signal sensors;

FIG. 10A is a top view of an example eyeglass frame and disengaged spring mounted temple electrodes;

FIG. 10B is a top view of an example eyeglass frame and partially engaged spring mounted temple electrodes;

FIG. 10C is a top view of an example eyeglass frame and engaged spring mounted temple electrodes;

FIG. 11A is a top view of a spring-pin assembly electrical contact design integrated into an example eyeglass frame;

FIG. 11B is another top view of a spring-pin assembly electrical contact design integrated into an example eyeglass frame;

FIG. 11C is a perspective view of a spring-pin assembly electrical contact design integrated into an example eyeglass frame;

FIG. 12A is a top view of an example eyeglass frame incorporating temple electrodes composed of elastomers, the elastomer temple electrodes being depicted in a disengaged position;

FIG. 12B is a top view of an example eyeglass frame incorporating temple electrodes composed of elastomers, the elastomer temple electrodes being depicted in an engaged position;

FIG. 13A is a top view of a flexible printed circuit board (PCB) assembly electrical contact design integrated into an example eyeglass frame, the flexible PCB board assembly being depicted in an engaged position;

FIG. 13B is a top view of a flexible PCB board assembly electrical contact design integrated into an example eyeglass frame, the flexible PCB board assembly being depicted in a disengaged position.

FIGS. 14 and 15 are a side view of an adjustable ear piece for an eyeglass frame according to some example embodiments.

FIGS. 16A and 16B are views of silver coated foam electrodes for an eyeglass frame according to some example embodiments.

FIG. 17 is a view of a hinge and a portion of an arm for an eyeglass frame in an open position according to some example embodiments.

FIG. 18 is a view of a hinge and a portion of an arm for an eyeglass frame in a closed position according to some example embodiments.

FIGS. 19, 20, 21 and 22 are views of hinges for an eyeglass frame according to some example embodiments.

FIG. 23 is a view of a hinge and a portion of an arm for an eyeglass frame in an open position according to some example embodiments.

FIG. 24 is a view of a hinge and a portion of an arm for an eyeglass frame in a closed position according to some example embodiments.

FIG. 25 is a view of a hinge and a portion of an arm for an eyeglass frame in an open position according to some example embodiments.

FIG. 26 is a view of a hinge and a portion of an arm for an eyeglass frame in a closed position according to some example embodiments.

These drawings depict aspects of example embodiments for illustrative purposes variations, alternative configurations, alternative components, and modifications may be made to these example embodiments.

### DETAILED DESCRIPTION OF THE EMBODIMENTS OF THE INVENTION

Reference will now be made in detail to some specific examples of the invention including the best modes contemplated by the inventors for carrying out the invention. Examples of these specific embodiments are illustrated in the accompanying drawings.

While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims. Also, it is to be understood that the phraseology and terminology employed herein are for the purpose of illustrative description and should not be regarded as limiting.

As the ways in which humans interact with computing devices change, computers may become usable for new purposes or may be specifically configured to be more efficient in performing existing tasks and resource usage. Embodiments described therein may involve measuring bio-signals such as brainwave patterns.

In the description, numerous specific details are set forth in order to provide a thorough understanding of the present invention. Particular example embodiments may be implemented without some or all of these specific details. In other instances, well known process operations have not been described in detail in order not to unnecessarily obscure the present invention.

Various techniques and mechanisms of the embodiments described herein will sometimes be described in singular form for clarity; the skilled reader will understand that such references include the plural form. Further, it should be noted that some embodiments may include multiple iterations of a technique or multiple instantiations of a mechanism unless noted otherwise. For example, a system uses a processor in a variety of contexts.

However, it will be appreciated that a system can use multiple processors. As an illustrative example, a eyewear device may include a processor that may in turn connect to a client device with another processor. It should also be noted that a processor may mean a multi-core processor. Furthermore, the techniques and mechanisms of the embodiments described herein will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory.

Consequently, a connection does not necessarily mean a direct, unimpeded connection unless otherwise noted.

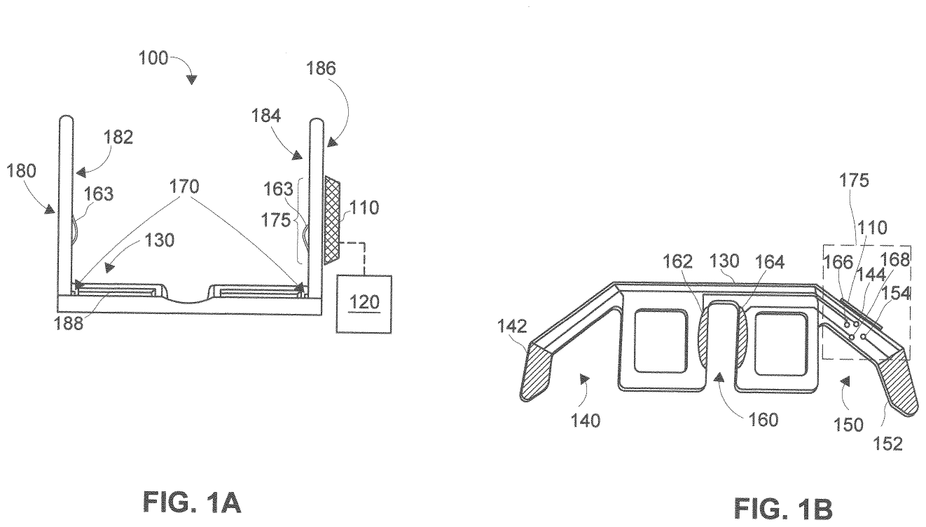

Referring to FIG. 1A, in accordance with an exemplary implementation of embodiments described herein there is provided a top view of an eyeglass frame 100 with a front portion 130, a signal pod 110 and a computing device 120. The signal pod 110 may be attachable to, and detachable from, an arm 186 of the eyeglass frame 100. The signal pod 110 may be in communication with the computing device 120 via, for example via Wi-Fi or Bluetooth. Bio-signal sensors may be included in the eyeglass frame 100 and may include electrically conductive surfaces (e.g., electrodes) and/or contacts that may be (e.g., 162, 163, 164, 142, 152) in contact with different portions of the user's read. These bio-signal sensors may be electrically isolated from each other and from the eyeglass frame 100 and, for example, may be printed on wiring in a non-conductive plastic frame. These bio-signal sensors may be electrically connected to a portion of an arm 140 of the eyeglass frame 100; the eyeglass frame 100 may be connected to the signal pod 110 such that the signal pod 110 may be in communication with the above-mentioned bio-signal sensors.

In some embodiments, the signal pod 110 may record measurements of voltage potential between the contacts of the bio-signal sensors (e.g., 142, 152 162, 163, 164) and the head of the user. The signal pod 110 may then transmit recorded measurements of voltage potential to the computing device 120 for processing into bio-signals (e.g., electroencephalograms, Electromyograms, or Electrooculograms).

Bio-signal sensors included in embodiments of the present disclosure may measure very small electrical signals emitted by the body, often as small as several micro-volts (millionths of a volt). Unfortunately, the human body may act as an antenna and may pick up electromagnetic interference, especially in the 50/60 Hz range. One of the signal interference reduction methods that may be applied by embodiments of the current disclosure is known as Driven Right Leg (DRL). The purpose of the DRL is to reduce common mode signals such 50/60 Hz AC line noise. The signal pod 110 may include one or more components to reduce signal noise, which may include: one or more instrument amplifiers that measure the potential difference across electrodes, one or more DRL circuits connected to one of the bio-signal sensors, one or more filters such as low pass for preventing aliasing and/or notch filters for reducing AC line noise, one or more analog to digital convertors, one or more wireless communication (e.g., Bluetooth) radios, and/or one or more batteries.

In an embodiment sensors usable with the eyeglass frame 100 may come in various shapes and be made of various materials. For example, the sensors may be partially made of a conductive material, including a conductive composite like rubber or conductive metal including metal plated or coated materials such as stainless steel, silver-silver chloride, and other materials. The sensors may include one or more bio-signal sensors, which may include EEG sensors, gyroscopes and accelerometers. The function of the sensors may be of various types, including: electrical bio-signal sensor in electrical contact with the user's skin; capacitive bio-signal sensor in capacitive contact with the user's skin. In some embodiments, some sensors like accelerometers, gyroscopes can be housed in the signal pod 110.

Referring to FIG. 1B, in accordance with the exemplary implementation of FIG. 1A, there is provided a front view of the eyeglass frame 100. As depicted in this exemplary implementation, the eyeglass frame 100 comprises the front portion 130 for holding two lenses, a first side arm 140, a second side arm 150, and a nose assembly 160. The first side arm 140 is connected to the front portion 130 and has a first side arm contact 142 in contact with a user's head where the first side arm contact 142 is electrically connected to a first side arm point 144. The second side arm 150 is connected to the front portion 130 opposite the first side arm 140 and having a second side arm contact 152 in contact with the user's head where the second side arm contact 152 is electrically connected to a second side arm point 154. The first side arm contact 142 and the second side arm contact 152 are positioned respectively on or within the first side arm 140 and the second side arm 150 such that the first side arm contact 142 and the second side arm contact 152 are, for example, positioned to be in contact with the ears of the user. The first side arm 140 may be either the right side arm or the left side arm of eyeglass frame 100 and such is similarly the case with the second side arm 140.

Further referring to the exemplary implementation of FIG. 1B, the nose assembly 160 seats the eyeglass frame on the user's nose and is connected to the front portion 130. The nose assembly 160 has a first nose contact 162 and a second nose contact 164 for supporting the front portion 130 on the user's nose where further the first nose contact 162 is electrically connected to a first nose point 166 and the second nose contact 164 is electrically connected to a second nose point 168. The first side arm contact 142, the first side arm point 144, the second side arm contact 152, the second side arm point 154, the first nose contact 162, the first nose point 166, the second nose contact 164, and the second nose point 168 may have, as a non-limiting example, electrically conductive surfaces such as conductive rubber with silver (or gold) compound coatings. Signals produced by the first side arm contact 142, the second side arm contact 152, the first nose contact 162 and the second nose contact 164 are electrically isolated from communicating or interfering with one another over the eyeglass frame 100. In some embodiments this may be accomplished by constructing the eyeglass frame of non-conducting plastic.

Further referring to the exemplary implementation of FIG. 1B, the electrical connections between: the first side arm contact 142, the second side arm contact 152, the first nose contact 162, and the second nose contact 164; and their corresponding connection to the first side arm point 144, the second side arm point 154, the first nose point 166 and the second nose point 168 may be implemented using embedded metal wiring (e.g., copper wire) in the non-conducting plastic eyeglass frame 100. Alternatively, where the eyeglass frame is metal or conducting, the contacts, points, and wiring may be electrically isolated from the eyeglass frame 100 with an insulation of known type. As non-limiting examples, wiring could be incorporated into embedded flexible circuit boards, or pad printed, silkscreened, or inkjet printed conductive ink.

Optionally, the first side arm 140 and the second side arm 150 may be pivotally hinged 170 to the front portion 130. The methods for the electrical connections to cross the hinges are well known in the art (not shown) and include, for example, flexible wiring. An attachment portion 175 for attachment of the signal pod 110 is depicted in FIG. 1A on an outer surface 186 of the second side arm 150. Alternatively, the attachment portion 175 for attachment of the signal pod 110 may be placed on any one (or more) of the top surface 188 of the front portion 130, the first outer surface 180 and the first inner surface 182 of the first side arm 140, and the second inner surface 184 of the second side arm 150 (not shown). The signal pod 110 may also include or integrate with a computing device in some example embodiments. The signal pod 110 may refer to the components integrating with eyeglass frame 100 to measure and capture bio-signal data from the user.

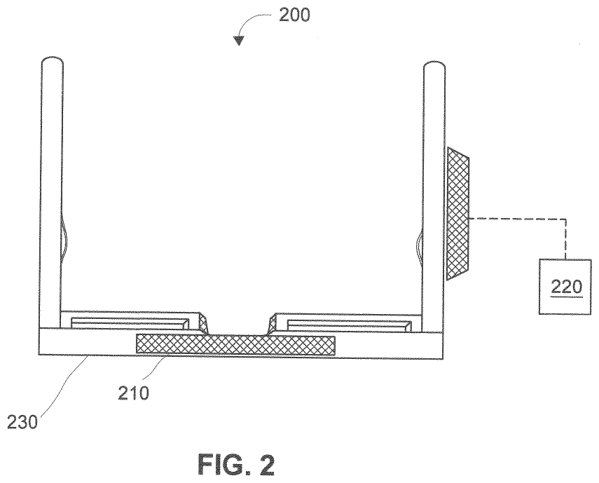

Referring to FIG. 2 in accordance with another exemplary implementation of embodiments described herein, there is provided a top view of an eyeglass frame 200 with a front portion 230, a signal pod 210 and a computing device 220. The signal pod 210 may be attachable and detachable to/from the front portion 230 of the eyeglass frame 200. The signal pod 210 may include various components and may also attach/detach to/from different portions of the eyeglass frame 200. The eyeglass frame 200 may have electrically conductive surfaces (electrodes) and/or contacts that may be designed to come into contact with a user's head. These contacts may be electrically isolated from one another and/or the eyeglass frame 200. These contacts may be connected to a portion of the front portion 230 of the eyeglass frame 200 and may be further connected to the signal pod 210. The signal pod 210 of FIG. 2 provide the same functionality as the signal pod 110 of FIGS. 1A and 1B, except that the signal pod 210 may be attachable to, and detachable from, the front portion 230. The signal pod 110 may be attachable to, and detachable from, an arm of the eyeglass frame 100. Any disclosure in respect of FIGS. 1A and 1B may also apply accordingly to FIG. 2 unless it does not logically apply, for example.

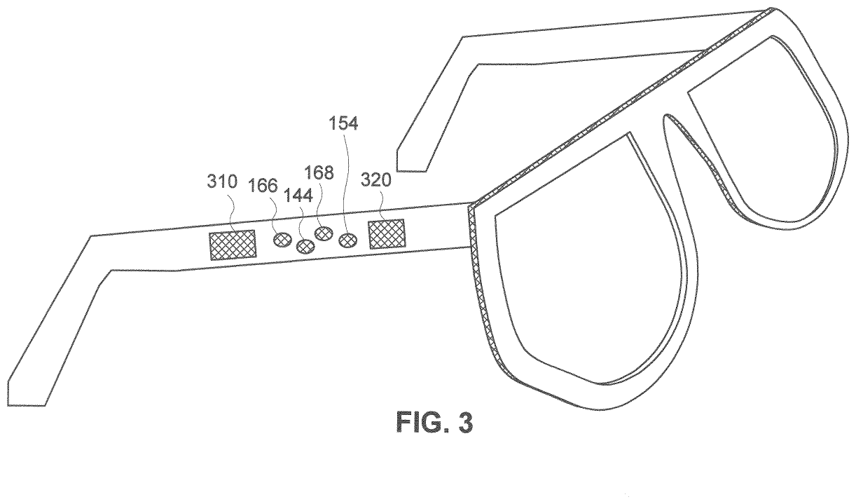

Referring to FIG. 3, there is provided a side view of example embodiment of an attachment portion of a side arm of eyeglass 100 for attachment of signal pod 110. The example embodiment depicted provides four contact points (144, 154, 166, 168) on a surface of the side arm: the first nose point 166, the first side arm point 144, the second nose point 168 and the second arm point 154. The example embodiment also depicts two magnetic stubs 310 and 320. The magnetic stubs 310 and 320 may be attachment points for corresponding magnetic stubs on the signal pod 110. The signal pod 110 may be attachable to and detachable from the eyeglass frame 100. The magnetic stubs may be composed of magnetic materials (e.g., iron, nickel, cobalt, their alloys, and some alloys of rare earth metals). Optionally, the two magnetic stubs 310 and 320 may be embedded within a non-conducting plastic eyeglass frame 100. Optionally, the eyeglass frame 100 may have teeth or slots (slots receive teeth or protrusions) for receiving corresponding slots or teeth of the signal pod 110 to ensure that the signal pod 110 is attached to the eyeglass frame 100 at a certain location. The location of the teeth or slot may, for example, correspond to the spacing and positioning of the magnetic stubs. There may be a protrusion or an indentation at the location of the two magnetic stubs 310 and 320.

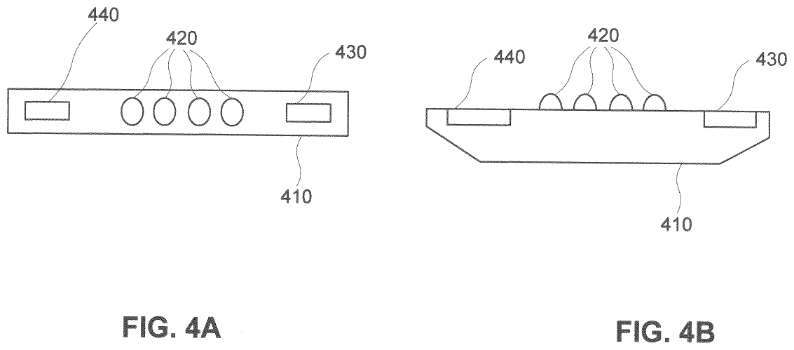

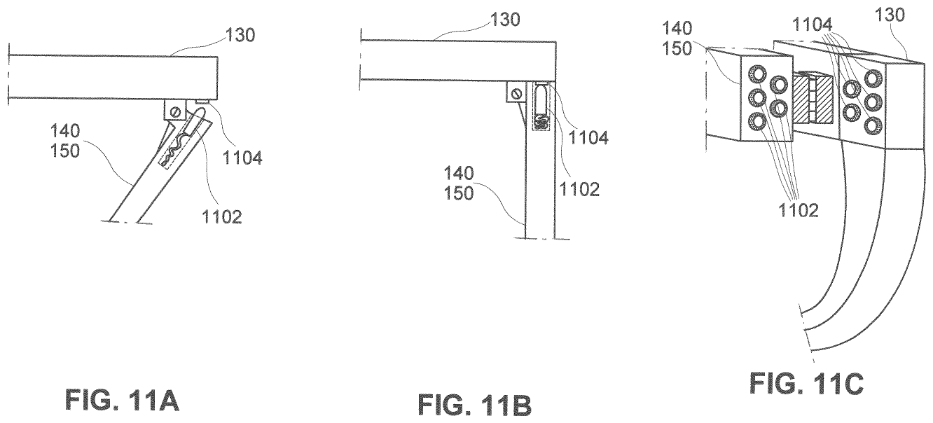

Referring to FIGS. 4A and 4B, there is provided a side view (FIG. 4A) and top view (FIG. 4B) of an example embodiment of the signal pod 110. The example signal pod 110 comprises a body 410, a number of spring loaded contacts 420 and a number of magnetic stubs 430 and 440. The body 410 of the example embodiment shown further comprises voltage potential sensors that may measure the potential between the four spring loaded contacts 420 and a radio (e.g., Wi-Fi or bluetooth) operable to transmit measured voltage potential data to the computing device 120 for processing to determine the brain states of the user 10. In some embodiments, the body 410 may further comprise a pod computing device for processing of voltage potential data (e.g., bio-signal measurements). Optionally, the four spring loaded contacts 420 may be pins that may fit with the first side arm point 144, the second side arm point 154, the first nose point 166, and/or the second nose point 168 where the points may be holes or indentations form fitting with the four spring loaded contacts 420. An example embodiment including spring loaded contacts 420 may be shown in FIG. 11.

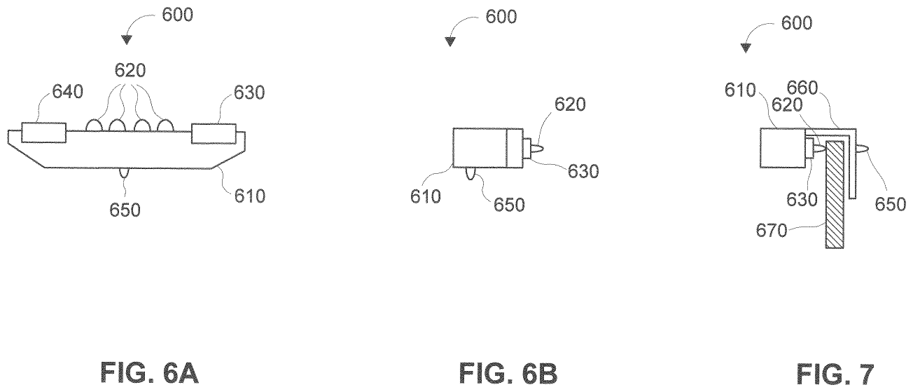

Referring to FIG. 6A, there is provided a top view of an example embodiment of a signal pod 600 in accordance with another exemplary implementation. The signal pod 600 may be similar to signal pod 110 and may comprise a body 610, a number of spring loaded contacts 620 and a number of magnetic stubs 630 and 640 depicted in FIG. 6A as protruding from the body 610 for fitting into corresponding indented slots on the eyeglass frame 100.

The following parts depicted in the figures may have the same or similar functions: the body 610 and the body 410; the four spring loaded contacts 420 and the four spring loaded contacts 620; the two magnetic stubs 430 and 440 and two magnetic stubs 630 and 640. The example signal pod 600 may further comprise a contact 650 for contacting with the user's head and providing an additional voltage potential measurement point. The signal pod 600, as shown, may be attachable to the inner surface 182 and/or the inner surface 184.

Referring to FIG. 6B, there is provided a side view of an example embodiment of the signal pod 600 configured for attachment to a top surface 188 of the front portion 130 of the eyeglass frame 100. The example signal pod 600 depicted in FIG. 6B comprises a body 610, spring loaded contacts 620, a magnetic stub 630 depicted as protruding from the body 610 for fitting into corresponding indented slots on the eyeglass frame 100, and spring loaded contact 650 (depicted at a right angle (approx. 90 degrees) to the spring loaded contacts 620) for fitting against the user's 10 forehead.

Referring to FIG. 7, there is provided a side view of an example embodiment of the signal pod 600 configured for attachment to an attachment portion on either the outer surface 180 or the outer surface 186 of the eyeglass frame 100. The signal pod 600 comprises the body 610, spring loaded contacts 620, magnetic stub 630 shown as protruding from the body 610 for fitting into corresponding indented slots on the eyeglass frame 100, and the spring loaded contact 650 on a U arm 660 to lift the spring loaded contact 650 over a side arm 670 of the eyeglass 100 to fit against the user's head where it makes electrical contact to the user's skin. This contact may be connected to the signal pod's 600 circuitry and may be an additional sensor for measuring biological signals. This component may also be attached to the front of the eyeglasses where pin 650 contacts the forehead of the user. Optionally, there may be more than one pins 650. The side arm 670 as shown may be a sectional view of the first side arm 140, second side arm 150 or any component corresponding to same, depending on whichever side arm the signal pod 600 is configured to be attached.

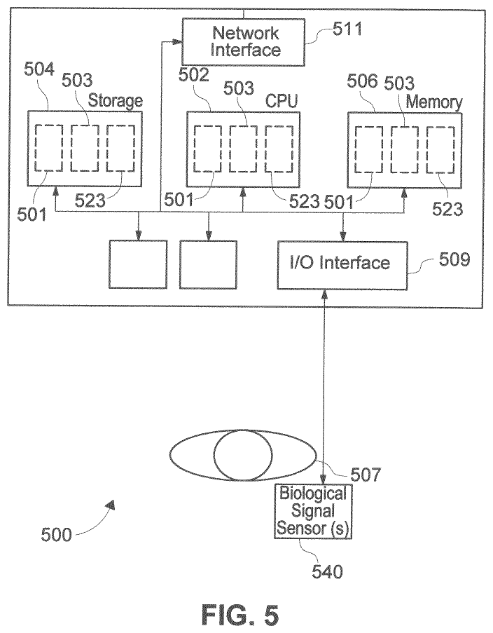

By way of example, FIG. 5 depicts a schematic view of an implementation of a computer device 500 that may include a central processing unit (“CPU”) 502 connected to a storage unit 504 and to a random access memory 506. The CPU 502 may process an operating system 501, application program 503, and data 523. The operating system 501, application program 503, and data 523 may be stored in storage unit 504 and loaded into memory 506, as may be required.

Biological signal sensors 540 may also be connected to the computer device 500, for example through I/O interface 509, and may be activated/deactivated or otherwise triggered through the I/O interface 509. Each biological signal sensor 540 may operate independently or may be linked with other biological signal sensor(s) 540, or linked to other computing devices. Each biological signal sensor 540 may transmit data to the CPU 502 (e.g., through the I/O interface 509).

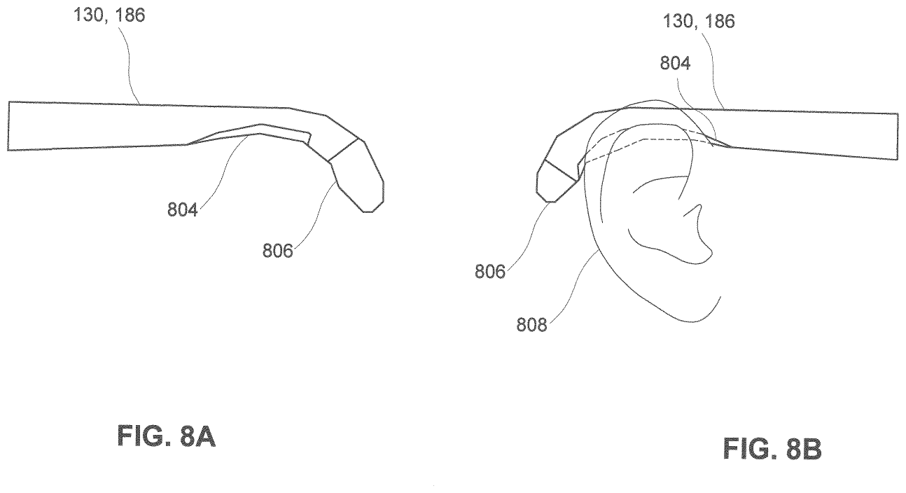

Referring to FIGS. 8A and 8B, there is provided a schematic view of bio-signal sensors 804, 806 integrated onto an arm 130, 186 of an eyeglass frame 100 according to an example embodiment. Although two bio-signal sensors 806, 804 are depicted, the skilled reader will understand that any number of bio-signal sensors may be incorporated in the manner depicted by the exemplary embodiment depicted in FIGS. 8A and 8B. The bio-signal sensors 804, 806 depicted in the exemplary embodiment of FIGS. 8A and 8B may be positioned within and/or atop the arm 130, 186 of eyeglass frame 100 such that, when a user 10 wears the eyeglass frames 100, said bio-signal sensors 804, 806 make contact with the ear 808 and or the head of user 10. Further, the bio-signal sensors 804, 806 may be intentionally positioned in order to ensure that bio-signal sensors 804, 806 are optimally oriented to detect bio-signals produced by user 10.

In some embodiments, the bio-signal sensors 804, 806 may incorporate conductive ink printed on the arm 130 of eyeglass frame 100, conductive rubber adhered or otherwise coupled to the arm 130 of eyeglass frame 100, and/or conductive fabric, metal, or another conductive material in order to facilitate bio-signal sensors 804, 806 detecting bio-signals produced by user 10. In an embodiment, bio-signal sensors 804, 806 may be constructed, in whole or in part, of a material that may deform in accordance with the contours of user's 10 ears 808 in order to ensure sufficient contact is established and/or maintained between bio-signal sensors 804, 806 and user 10.

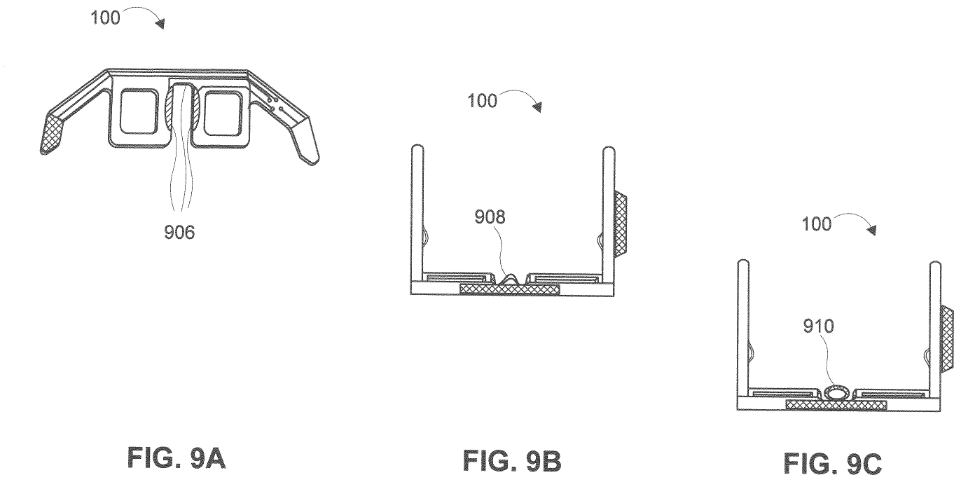

Referring to FIG. 9A, there is provided a schematic view of an example eyeglass frame 100 and example orientations of attached bio-signal sensors 906, 908, 910 around the user's 10 nose bridge according to an example embodiment. FIG. 9A provides a rear view of eyeglass frame 100 depicting three nose bridge sensors positioned to effect contact with the nose of user 10 laterally at either side of user's 10 nose, and/or perpendicularly in the nose bridge region. In some embodiments, bio-signal sensors 906, 908, 910 positioned on the eyeglass frame 100 in order to produce contact with the nose region of a user 10 may be constructed in a fashion (structurally and/or materially designed) in order to ensure sufficient contact is established and maintained with the user's 10 nose region without producing discomfort or causing eyeglass frame 100 to shift forward away from the nose region of user 10.

FIG. 9B provides an alternative design of an eyeglass frame 100 with attached arc-shaped bio-signal sensor 908 positioned horizontally thereon in order to establish contact with the user's 10 nose bridge region according to an embodiment. Bio-signal sensor 908 may be constructed to incorporate conductive ink printed on the outer surface of bio-signal sensor 908, conductive rubber adhered or otherwise coupled to the outer surface of the bio-signal sensor 908, and/or conductive fabric, metal, or another conductive material in order to facilitate bio-signal sensor 908 detecting bio-signals produced by user 10.

FIG. 9C provides an alternative design of an eyeglass frame 100 with attached circular-shaped bio-signal sensor 910 positioned horizontally thereon in order to establish contact with the user's 10 nose bridge region according to an embodiment. Bio-signal sensor 910 may be constructed to incorporate conductive ink printed on the outer surface of bio-signal sensor 910, conductive rubber adhered or otherwise coupled to the outer surface of the bio-signal sensor 910, and/or conductive fabric, metal, or another conductive material in order to facilitate bio-signal sensor 908 detecting bio-signals produced by user 10.

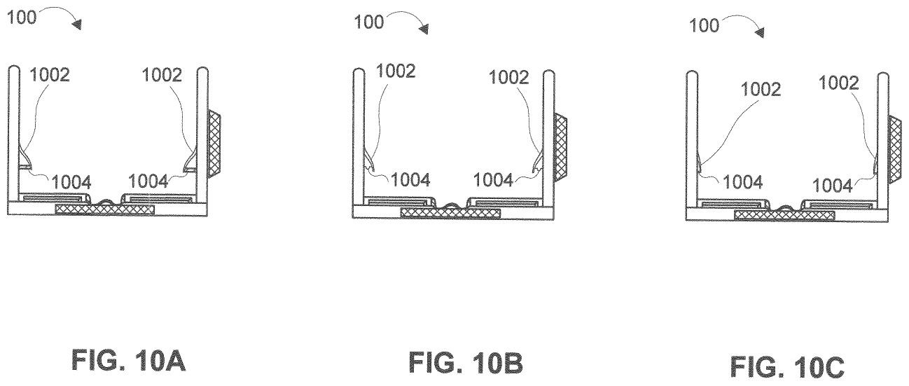

Referring now to FIGS. 10A, 10B, and 10C, there is provided a set of top views of an example eyeglass frame 100 and spring mounted temple electrodes 1002 according to some embodiments. FIG. 10A provides a top view of an example eyeglass frame 100 with example temple electrodes 1002 and temple electrode springs 1004 attached to the eyeglass frame 100 at the first inner surface 182 of the first side arm 140, and/or the second inner surface 184 of the second side arm 150. Temple electrodes 1002 may comprise bio-signal sensors that may be constructed to incorporate conductive ink printed on the outer surface of temple sensor 1002, conductive rubber adhered or otherwise coupled to the outer surface of temple electrode 1002, and/or conductive fabric, metal or another conductive material in order to facilitate temple electrode 1002 detecting bio-signals produced by user 10.

The temple electrodes 1002 depicted in FIG. 10A are represented in a disengaged position and, thus, the temple electrode spring 1004 is in its fully extended position. FIG. 108 depicts the temple electrodes in a semi-engaged position (e.g., in the case of a user 10 who has only partially completed putting on eyeglass frame 100), and thus, temple electrode spring 1004 is depicted in a semi-compressed position. FIG. 10C depicts the temple electrodes 1002 in a fully-engaged position (e.g., the user 10 has successfully completed putting on eyeglass frame 100), and thus, temple electrode spring 1004 is depicted in its fully-compressed position. When in the fully engaged position, the pressure established between temple electrodes 1002 and the head of user 10 may establish conductive contact between the head of the user 10 and temple electrode 1102 and may, thus, place bio-signal sensors within temple electrode 1102 in an optimal position to detect bio-signals produced by user 10.

In some embodiments, the temple electrode 1002 may be constructed out of rigid or semi-rigid materials and temple electrode spring 1004 may produce even pressure as it compresses in order to ensure consistent contact. The even pressure produced by the compression of temple electrode spring 1004 may also serve to ensure that eyeglass frame 100 and associated components remain positionally secure on the head of user 10. For example, were temple electrode spring 1004 to provide inadequate pressure between temple electrode 1002 and the head of user 10, temple electrode 1002 may shift positionally on the head of user 10 and temple electrode 1002 may:

a) cause discomfort to user 10; and
b) produce less effective sensor readings from bio-signals within temple electrode 1002.

In some embodiments, temple electrode 1002 and/or temple electrode spring 1104 may be constructed of conductive compressible foam and/or a combination of spring and/or elastomer materials. In some embodiments temple electrode 1102 may be constructed of a flexible or rigid material. In some embodiments, temple electrode 1102 may be removable and may be replaced with temple electrodes 1102 or temple electrode springs 1104 of various tensions, designs, and/or materials.

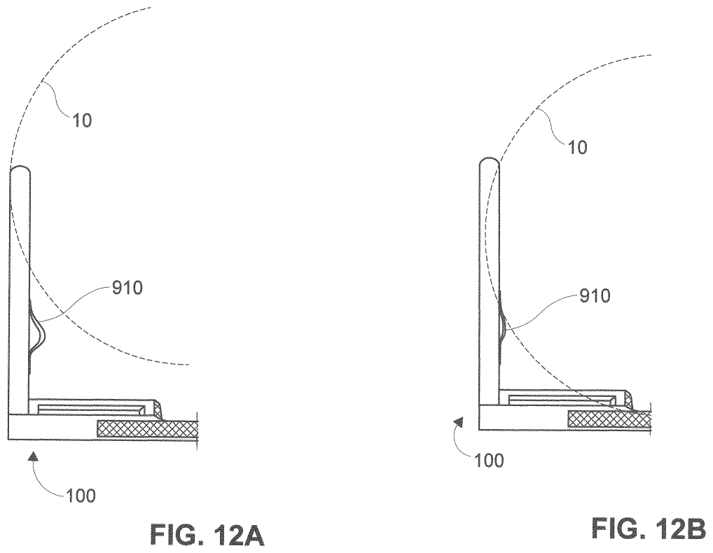

FIG. 12A and FIG. 12B further illustrate temple electrodes 910 in a disengaged position (see FIG. 12A) and engaged position (see FIG. 12B) according to some embodiments. The temple electrodes 910 depicted in FIG. 12A and FIG. 12B may be constructed of an elastomer material and may be constructed of and/or coated or covered in a conductive material in a similar fashion described above in reference to FIG. 11.

FIG. 12A depicts temple electrode 910 in a disengaged position as user 10 has not yet fully placed eyeglass frame 100 onto his or her head. As no pressure between the head of user 10 and the temple electrode 910 has yet been established, temple electrode 910 remains in its resting position and conductive contact between temple electrode 910 and user 10 is not established.

FIG. 12B depicts temple electrode 910 in an engaged position as user 10 has fully placed eyeglass frame 100 onto his or her head. The pressure established between temple electrode 910 and the head of user may establish conductive contact between the head of the user 10 and temple electrode 910 and may, thus, place bio-signal sensors in temple electrode in an optimal position to detect bio-signals produced by user 10.

FIGS. 11A, 11B, and 11C provide views of a spring-pin assembly electrical contact 1104-1102 design integrated into an example eyeglass frame according to some embodiments. Conductive wiring may provide for communicative contact between the various components described in the present disclosure (e.g., signal pod 130, temple electrodes 1102, and/or bio-signal sensors 804, 806, 906, 908, 910, etc.). Conductive wiring may be incorporated into the construction of eyeglass frame by various means. For example, some embodiments may use conductive wiring printed onto the surface of eyeglass frame 100 and coated with a non-conductive material. Some embodiments may use conductive wiring printed onto a flexible substrate and moulded within the material composing eyeglass frame 100. Some embodiments may use discrete conductive wiring within the eyeglass frame 100. Some embodiments may incorporate conductive wiring that has been screen printed or 3D printed onto the eyeglass frame 100 with conductive ink (e.g., silver ink). All of these example embodiments have their own, particular, advantages. However, as the majority of eyeglass frame arms (e.g., 140, 150) are attached to the eyeglass frame front portion 130 with a hinge mechanism, some means of establishing and maintaining contact between conductive wiring in, or on, the eyeglass frame's front portion 130 and the eyeglass frame arms (e.g., 140, 150) is required.

FIGS. 11A, 11B, and 11C provide an example embodiment of a spring-pin assembly electrical contact 1104-1102 design integrated into an example eyeglass frame in order to establish and maintain contact between wiring of components in or on the eyeglass frame 100. FIG. 11A depicts a top view of eyeglass frame 100 with a spring pin 1102 embedded in a channel within eyeglass frame arm 140 and/or 150, and a spring pin assembly contact attached to the front position 130 of eyeglass frame 100. In FIG. 11A the eyeglass frame 100 and eyeglass frame arm 140 and/or 150 are depicted in the open position, thus the spring pin 1102 is in its extended position but is not making conductive contact with the spring pin assembly contact 1104.

FIG. 11B depicts a top view of eyeglass frame 100 with a spring pin 1102 embedded in a channel within eyeglass frame arm 140 and/or 150, and a spring pin assembly contact 1104 attached to the front portion 130 of eyeglass frame 100. In FIG. 11B the eyeglass frame 100 and eyeglass frame arm 140 and/or 150 are depicted in the closed position, thus the spring pin 1102 is in its active position and is maintaining conductive contact with the spring pin assembly contact 1104.

FIG. 11C depicts a perspective view of eyeglass frame 100 with multiple spring pins 1102 embedded in channels within eyeglass frame arm 140 and/or 150, and multiple spring pin assembly contacts 1104 attached to the front portion 130 of eyeglass frame 100. Multiple components of eyeglass frame 100 may require communicative contact with one another. Thus, the spring pin assembly solution depicted in FIGS. 11A, 11B, and 11C may allow for multiple spring pins and multiple spring pin assembly contacts to be incorporated into the front portion 130 and eyeglass frame arm 140 and/or 150 of eyeglass frame 100. Further, solutions such as flexible wiring suffer from degradation of conductive wiring over time to due to wear and tear. The solution depicted above may establish and maintain conductive contact while minimising wear and tear on conductive components. Further, design aesthetic is an extremely important factor in the design of eyeglasses, the solution depicted above may be incorporated into eyeglasses and may allow manufacturers to maintain their desired design aesthetic.

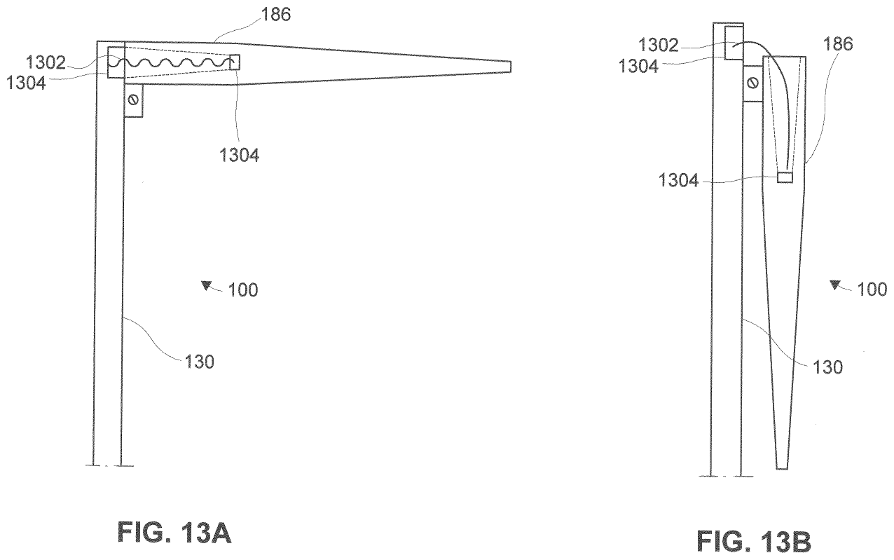

FIGS. 13A and 13B provide an example embodiment of a flexible PCB board assembly electrical contact design integrated into an example eyeglass frame 100 in order to establish and maintain conductive contact between wiring of components in or on the eyeglass frame 100 according to some embodiments. FIG. 13A depicts a top view of a flexible PCB board assembly 1302 housed within a channel in eyeglasses frame arm 140 and/or 150. As the eyeglass frame 100 is depicted in a fully opened position (e.g., placed on the head of a user 10) the ends of flexible PCB board assembly 1302 are making conductive contact with flexible PCB board assembly contacts 1304 on the front portion of eyeglass frame 130 and at the base of the channel in eyeglass frame arm 140 and/or 150. Once the flexible PCB board assembly 1302 establishes and maintains contact with the flexible PCB board assembly contacts 1304, conductive contact may be established enabling components in or on the eyeglass frame 100 to communicate with one another via wires embedded therein.

FIG. 13B depicts a top view of a flexible PCB board assembly 1302 housed within a channel in eyeglasses frame arm 140 and/or 150 according to some embodiments. As the eyeglass frame 100 is depicted in an open position, the ends of flexible PCB board assembly 1302 are not making contact with both flexible PCB board assembly contacts 1304. As a result, conductive contact may not be established between components in or on the eyeglass frame 100 and said components may not be able to communicate with one another via wires embedded therein.

As with the spring pin assembly solution described above, the flexible PCB board assembly solution depicted in FIGS. 13A, and 13B may allow for conductive contacts to be incorporated into the front portion 130 and eyeglass frame arm 140 and/or 150 of eyeglass frame 100. Further, known solutions such as flexible wiring may suffer from degradation of conductive wiring over time to due to wear and tear. The solution depicted above may establish and maintain conductive contact while minimising wear and tear on conductive components. Further, design aesthetic is an extremely important factor in the design of eyeglasses, the solution depicted above may be incorporated into eyeglasses and may allow manufacturers to maintain their desired design aesthetic.

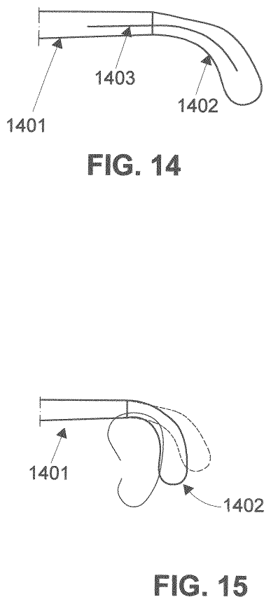

FIGS. 14 and 15 are a side view of an adjustable ear piece for an eyeglass frame according to some example embodiments. The adjustable ear piece both secures the eyewear and provides sufficient pressure to the electrode. In some example embodiments, the eyeglass frame may have conductive contacts in the adjustable ear piece. The adjustable ear piece connects to a side arms of the eyeglass frame and has head conductive contacts in contact with a user's head and ears when in use. The conductive contacts are electrically connected to the side arms and a signal pod. The adjustable ear piece is formable or malleable to conform to the user's head and ears. The adjustable ear piece has a formable component that is conductive or coated in conductive material and an internal malleable metal piece that can bend or reshape.

The adjustable ear piece has a main part of the arm 1401 made of a rigid plastic or metal or other material for this example. The adjustable ear piece has a soft rubber or elastomer or elastomer foam ear piece 1402 which is either conductive or coated in a conductive material. Internal to the adjustable ear piece there is a malleable metal piece 1403 which can be bent to reshape the foam ear piece 1402 to conform to various head and ear shapes and provide a good fit and consistent connection between the conductive element and the skin. The ear piece is adjustable in different directions and dimensions to conform to the head and ear of the user.

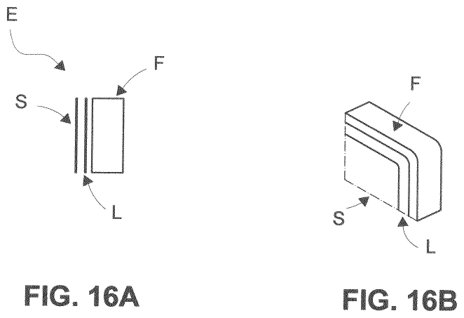

In some example embodiments, the adjustable ear piece has silver coated foam electrodes. FIGS. 16A and 16B are views of silver coated foam electrodes E for an eyeglass frame according to some example embodiments. The silver coated foam electrodes include a flexible foam F, or memory foam which has a layer L of rubber paint, or TPU film, or other smooth or flexible or stretchable substrate applied to it. Then a layer of silver ink S is applied to the smooth substrate. Alternatively the silver ink could be applied to a film which is then applied to the foam. Another option is to apply the silver ink to a flexible film which is then placed in a mold and the foam is molded behind it.

For some embodiments described herein, a front portion of the eyeglass frame refers to a part or portion of the eyeglasses that contain lenses and nose piece. The arm may refer to the part of the eyeglasses which extends from front portion of the frame to the ears of the user. The hinge may connect the eyeglass frame to the arm of the eyeglasses. The hinge enables the arm to move between an open position and a closed position. An open position for eyeglasses may refer to a position where the arms are folded out (or substantially folded out) such that they are ready to put on the head of the user to wear. A closed position for eyeglasses may refer to a position where the arms are folded up or in towards the eyeglass frame for storage, for example. In some example embodiments, the hinge has a flex portion foldable when the side arms move between the open position and the closed position.

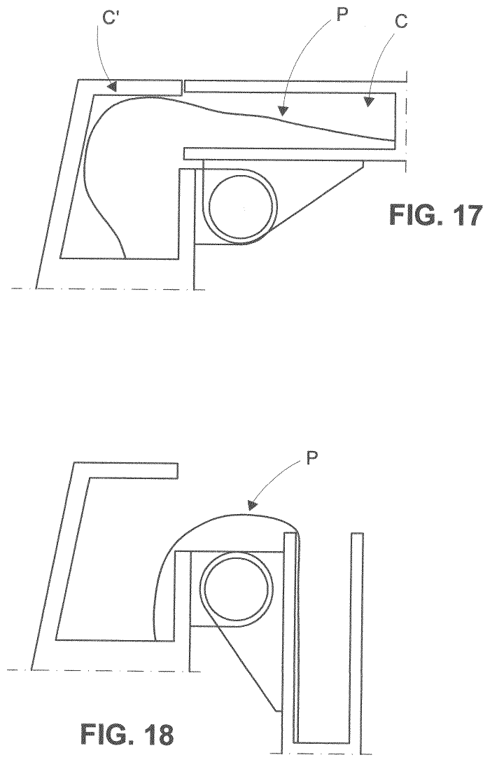

FIG. 17 is a view of a hinge and a portion of an arm for an eyeglass frame in an open position according to some example embodiments. FIG. 18 is a view of a hinge and a portion of an arm for an eyeglass frame in a closed position according to some example embodiments. When the arm is in an open position a Flex PCB portion P is pushed into the outside of the cavity C,C′ in the arm and frame of the eyeglasses, respectively. When the arm is in the closed position the flex PCB portion P is pulled taut. The Flex PCB portion could be a flexible PCB or any flexible substrate with electrically conductive traces printed or otherwise adhered to it.

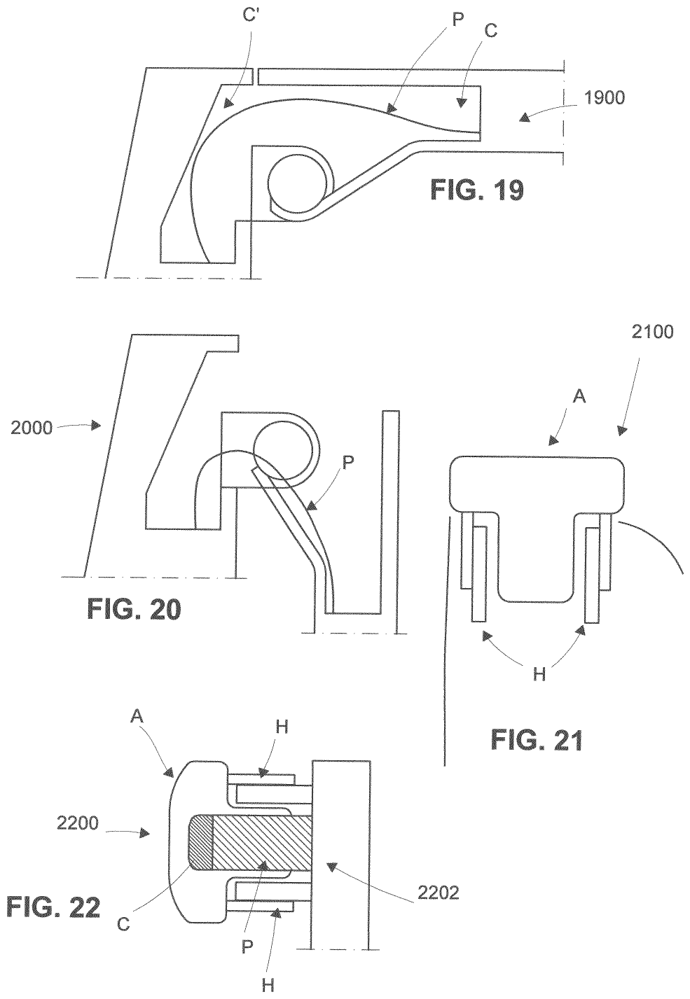

FIGS. 19, 20, 21 and 22 are views of hinges for an eyeglass frame according to some example embodiments. In the example configuration shown, the hinge H of the eyeglass is split in two sides to allow the Flex PCB portion P to go through the middle of it. Reference 1900 shows where the Flex PCB portion that sits when the arm is open. Reference 2000 shows where Flex PCB portion P sits when the arm A is closed. Note that the Flex PCB portion P moves through the axis of the hinge. Reference 2100 shows the arm A in the open position from the back. Reference 2200 shows the arm in the closed position from the side and the shaded area 2202 is the Flex PCB portion P where the hinge does not get in its way.

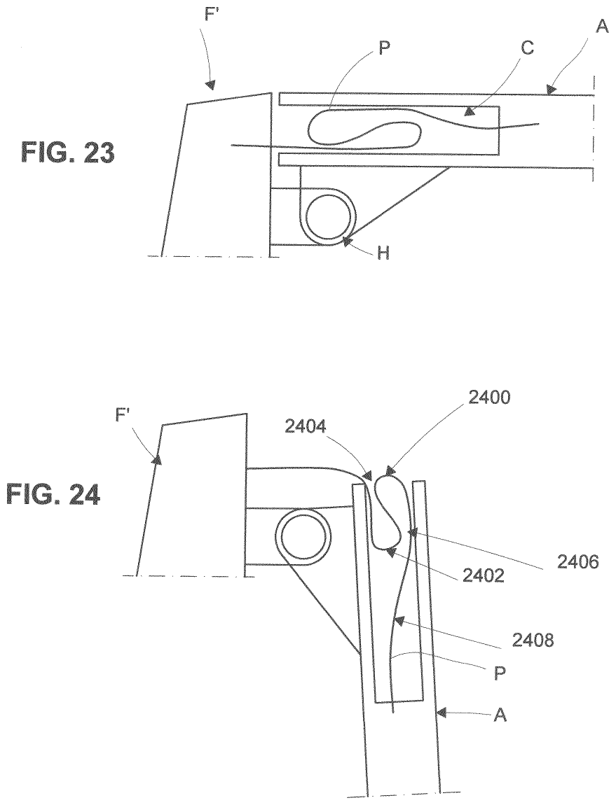

FIG. 23 is a view of a hinge and a portion of an arm A for an eyeglass frame F′ in an open position according to some example embodiments. FIG. 24 is a view of a hinge H and a portion of an arm A for an eyeglass frame in a closed position according to some example embodiments.

In this configuration the flex PCB portion P folds into a cavity just in the arm. The folds are constrained in the cavity C such that when the arm A is in the fully closed position the flex PCB portion pulls the two folds closer together. The outer fold 2400 does not get pulled out of the cavity and the inner fold 2402 does not get within one fold radius of the outer fold. By having the Flex PCB portion exit the cavity on the hinge side, the part of the flex moving into the cavity moves together with the outer fold 2400 at point 2404. The inner fold 2402 rolls along the outer fixed portion of the Flex PCB portion 2408 at point 2406. This guides the Flex PCB portion back into the cavity and reduces the force needed to push it in. There is no cavity in the frame portion of the eyeglasses in this example embodiment.

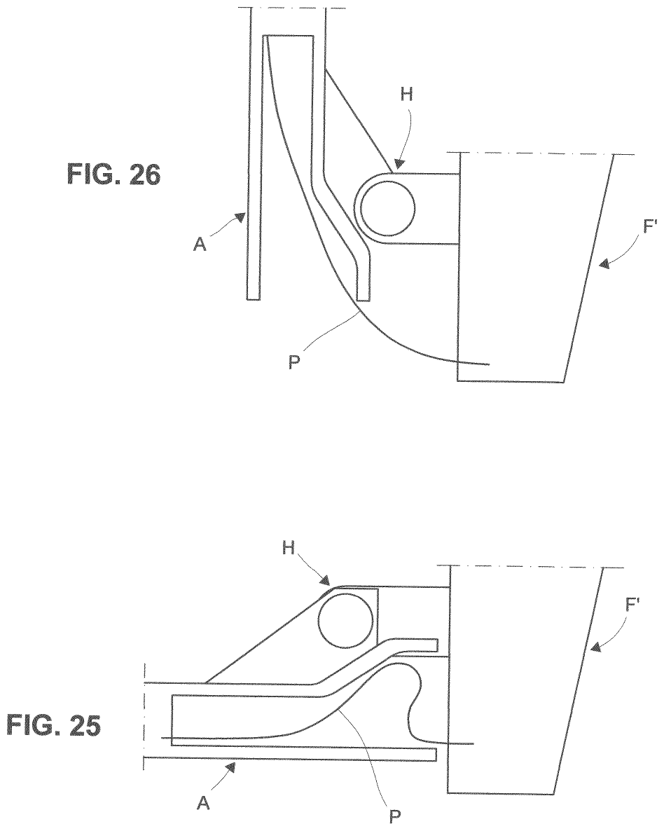

FIG. 25 is a view of a hinge H and a portion of an arm A for an eyeglass frame F′ in an open position according to some example embodiments. FIG. 26 is a view of a hinge and a portion of an arm for an eyeglass frame in a closed position according to some example embodiments. In this configuration the flex PCB folds back on itself inside the arm.

It will be appreciated that any module or component exemplified herein that executes instructions may include or otherwise have access to computer readable media such as storage media, computer storage media, or data storage devices (removable and/or non-removable) such as, for example, magnetic disks, optical disks, tape, and other forms of computer readable media. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD), blue-ray disks, or other optical storage, magnetic cassettes, magnetic tape, magnetic disk storage or other magnetic storage devices, or any other medium which can be used to store the desired information and which can be accessed by an application, module, or both. Any such computer storage media may be part d the mobile device, tracking module, object tracking application, etc., or accessible or connectable thereto. Any application or module herein described may be implemented using computer readable/executable instructions that may be stored or otherwise held by such computer readable media.

Thus, alterations, modifications and variations can be effected to the particular embodiments by those of skill in the art without departing from the scope of this disclosure, which is defined solely by the claims appended hereto.

In further aspects, the disclosure provides systems, devices, methods, and computer programming products, including non-transient machine-readable instruction sets, for use in implementing such methods and enabling the functionality described previously.

Although the disclosure has been described and illustrated in exemplary forms with a certain degree of particularity, it is noted that the description and illustrations have been made by way of example only. Numerous changes in the details of construction and combination and arrangement of parts and steps may be made. Accordingly, such changes are intended to be included in the invention, the scope of which is defined by the claims.

Except to the extent explicitly stated or inherent within the processes described, including any optional steps or components thereof, no required order, sequence, or combination is intended or implied. As will be understood by those skilled in the relevant arts, with respect to both processes and any systems, devices, etc., described herein, a wide range of variations is possible, and even advantageous, in various circumstances.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the present discussion, numerous references are made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The present discussion provides many example embodiments. Although each embodiment represents a single combination of inventive elements, other examples may include all possible combinations of the disclosed elements. Thus if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

For simplicity only one computing device 500 is shown but system may include more computing devices 500 operable by users to access remote network resources and exchange data. The computing devices 500 may be the same or different types of devices.

The computing device 500 comprises at least one processor 502, a data storage device 504 (including volatile memory or non-volatile memory or other data storage elements or a combination thereof), and at least one communication interface.

For example, and without limitation, the computing device 500 may be a server, network appliance, set-top box, embedded device, computer expansion module, personal computer, laptop, personal data assistant, cellular telephone, smartphone device, UMPC tablets, video display terminal, gaming console, and wireless hypermedia device or any other computing device capable of being configured to carry out the methods described therein.

FIG. 5 is a schematic diagram of computing device 500, exemplary of an embodiment. As depicted, computing device 500 includes at least one processor 502, memory 504, at least one I/O interface 506, and at least one network interface 511.

Each processor 502 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 504 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each network interface 511 enables computing device 500 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Computing device 500 is operable to register and authenticate users 507 (using a login, unique identifier, and password for example) prior to providing access to applications, a local network, network resources, other networks and network security devices. Computing devices 500 may serve one user or multiple users.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only.

1. An eyeglass frame comprising:
a front portion (130) for holding two lenses;
an assembly for attaching to a signal pod (110);
side arms (140, 150) connected to the front portion (130);
a nose assembly (160) connected to the front portion (130) and having nose contacts (162, 164) for supporting the front portion (130) on a user's nose wherein the nose contact (162, 164) is electrically connected to the signal pod (110); an adjustable ear piece (1402) connected to each of the side arms and having head contacts in contact with a user's head and ears, wherein the head contacts are electrically connected to the side arms (140, 150) and the signal pod (110), the adjustable ear piece being formable or malleable to conform to the user's head and ears.

2. The eyeglass frame of claim 1 wherein the adjustable ear piece has a formable component that is conductive or coated in conductive material and an internal malleable metal piece that can bend or reshape.

3. The eyeglass frame of claim 1 wherein the adjustable ear piece comprises silver coated foam electrodes.

4. The eyeglass frame of claim 1 further comprising a flex PCB portion and a hinge pivotally (170) connecting the side arms (140, 150) to the front portion (130) to move the side arms between an open position and a closed position, the flex PCB portion foldable when the side arms (140, 150) move between the open position and the closed position.

5. An adjustable ear piece for an eyeglass frame, the adjustable ear piece connecting to a side arm (140, 150) of an eyeglass frame and having head contacts in contact with a user's head and ears, wherein the head contacts are electrically connected to the side arm (140, 150) and to a signal pod (110), the adjustable ear pieces being formable or malleable to conform to the user's head and ears.

6. The adjustable ear piece of claim 5 comprising a formable component that is conductive or coated in conductive material and an internal malleable metal piece that can bend or reshape.

7. The adjustable ear piece of claim 5 comprising silver coated foam electrodes.

8. A hinge for an eyeglass frame configured to pivotally connect a side arm (140, 150) to a front portion (130) of an eyeglass frame to move the side arm between an open position and a closed position, the hinge having a flex PCB portion foldable when the side arm (140, 150) move between the open position and the closed position.

9. The eyeglass frame of claim 1, wherein the adjustable ear piece (1402) has a soft rubber or elastomer component which is conductive or is coated in a conductive material.
